(19) United States
(12) Patent Application Publication
(10) Pub. No.: US 2010/0152640 A1
(43) Pub. Date: Jun. 17, 2010

(54) METHODS AND APPARATUS FOR VASCULAR ACCESS

(75) Inventors: Arthur L. Golding, Los Angeles, CA (US); Rick Berglund, San Diego, CA (US); Eric Warner, Oceanside, CA (US)

Correspondence Address: MAYER & WILLIAMS PC, 251 NORTH AVENUE WEST, 2ND FLOOR, WESTFIELD, NJ, 07090, US

(73) Assignee: IMTECBIOMEDICAL, INC., San Diego, CA (US)

(21) Appl. No.: 12/555,608

(22) Filed: Sep. 8, 2009

Related U.S. Application Data
(63) Continuation-in-part of application No. 12/206,674, filed on Sep. 8, 2008.
(60) Provisional application No. 61/094,745, filed on Sep. 5, 2008.
(60) Provisional application No. 61/216,821, filed on May 21, 2009.

(51) Int. Cl. A61M 1/14

(52) U.S. Cl. 604/6.16

(57) ABSTRACT

Methods and systems are described for creating chronic vascular access or hemodialysis. These methods and systems eliminate the insertion of needles which are now required for using the available methods of obtaining vascular access. Thus multiple complications due to needle insertion through skin, tissue, and vein or graft wall are prevented. A low-profile stable transcutaneous implant's external surface and stability minimizes infection. The implant is readily joined to a connector device that functions substantially automatically and which provides high blood flow volumes for hemodialysis.

### CROSS-REFERENCE TO RELATED APPLICATIONS

This application claims the benefit of U.S. Provisional Patent Application Ser. Nos. 61/094,745, filed Sep. 5, 2008, entitled “Methods and Apparatuses for Conducting Dialysis”, and 61/216,821, filed May 21, 2009, entitled “Method and Device For Vascular Access”. This application is also a continuation-in-part of U.S. patent application Ser. No. 12/206,674, filed Sep. 8, 2008, entitled “Method And Device For Dialysis”. All of the above applications are herein incorporated by reference in their entirety.

### BACKGROUND

The use of hemodialysis to maintain the lives of patients suffering from kidney failure was initiated in the 1960′s. It has become a widely used medical technology for patients of all ages suffering from multiple disease states that produce severe damage to the kidneys. This damage prevents the normal excretion of the toxic products of metabolism and without the use of dialysis, death will occur within a short period of time.

Hemodialysis requires the use of a dialysis machine that filters the toxic substances from the patient's blood on a regular basis—generally three times each week on an every-other-day schedule for a duration of approximately four hours for each treatment.

In order to successfully perform chronic hemodialysis it is necessary to have chronic access to the patient's circulatory system (vascular access). A hemodialysis treatment includes simultaneously withdrawing and infusing over 500 cc per minute of blood from and to the patient utilizing one of three forms of vascular access.

In one system, a central venous catheter (CVC) is placed within a large central vein and is generally used to institute temporary emergency or urgent hemodialysis. It provides limited blood flow, causes stenosis and thrombosis of central veins, and frequently becomes non-functional due to clotting and is often the site for local and bloodstream infections. The use of the CVC may be the least desirable of the present methods for permanent vascular access.

In another system a autologous arterio-venous fistula (AVF) is used which requires the presence of an adequately-sized undamaged artery and vein in an extremity. The blood vessels must be in close proximity and must be able to be anastomosed (joined) to create a dilated venous system through which flows a large volume of arterial blood. The markedly increased blood flow at increased pressure in the dilated vein is then accessed by the placement of two (2) large bore needles. These needles provide flow to and from the dialysis machine. Unfortunately, less than 50% of patients requiring hemodialysis have adequate blood vessels for the creation of a successful AVF. The increased flow and pressure within the dilated vein and the repeated insertion of needles through the skin, subcutaneous tissues and the vein wall eventually causes damage to the vein resulting in stenosis, aneurysms and thrombosis of the AVF. Nonetheless, it is currently considered the “best” method of vascular access for hemodialysis.

In yet another system, an interposition graft arterio-venous fistula (IGAVF) is used when the patient does not have adequate veins for construction of an AVF. A tubular graft 6 to 7 mm in diameter, generally composed of polytetrafluroethylene (PTFE), and 25 to 40 cm in length is anastomosed to an adequately-sized extremity artery and vein and tunneled immediately beneath the skin surface. Arterial blood flows thru the graft into the venous system at high flows and pressure and, as in the AVF system, two (2) large bore needles must be inserted into the graft to perform hemodialysis. These needles result in repeated damage to the skin, subcutaneous tissues and graft wall, producing stenosis, false aneurysms, bleeding, clotting of the graft, and local and blood stream infection.

Each patient with an AVF or IGAVF has to undergo the insertion of a minimum of 312 needles each year and often many more insertions, due to difficult or improper placement, in order to perform hemodialysis. Insertion of the needles at the start of hemodialysis requires skill, is painful, and results in anxiety for the patient. Removal of the needles at the completion of hemodialysis requires prolonged pressure at the puncture sites to induce clot formation and tissue coaptation to prevent bleeding.

Improper needle placement results in local bleeding into the tissues (e.g., hematoma formation) and can produce false aneurysms, which are spaces within the tissues surrounding the puncture sites filled with flowing blood under high pressure. The average dialysis patient undergoes two (2) operative procedures each year to repair or create new or revise prior vascular access.

Approximately thirty years ago the problems associated with repeated use of needles in patients using IGAVF for hemodialysis were recognized. A device was developed and released by Bentley Laboratories of Irvine, Calif., termed the Bio-Carbon Vascular Access Prosthesis—consisting of a permanent percutaneous port attached to a PTFE graft. The PTFE graft was inserted in the same manner as an IGAVF and the port brought through the subcutaneous tissue and skin for permanent vascular access. The device eliminated the need for needle insertion by using the percutaneous port and a connecting device as the means of providing blood inflow to and outflow from the dialysis machine. The Bentley system received FDA approval and was used in a significant number of patients worldwide and reported on in the peer-reviewed medical literature. It provided adequate blood flow rates for hemodialysis and eliminated the problems associated with the use of needles. Its major drawbacks were the formation of a sinus tract surrounding the port's exit site through the skin, a site for local infection to develop, and a cumbersome connector for connecting to the dialysis machine. Nonetheless, patients, dialysis nurses and nephrologists were enthusiastic in its use. However, the manufacturer of the device discontinued its production in the early 1980's.

In addition to the Bentley device, a device was developed, e.g., a PTFE graft attached to a permanent port containing a silastic “plug” through which needles were inserted for dialysis. This device suffered from leakage through the silastic material, recirculation, sinus tract formation, and clot formation within the device.

The three present methods described above for providing vascular access for hemodialysis have been in use for over 40 years. Other than attempting to influence nephrologists and surgeons to create an AVF or its several modifications, or using modified designs or materials for CVCs and IGAVFs, no new methods of creating permanent vascular access for hemodialysis are available.

The maintenance of permanent and adequate vascular access for hemodialysis with minimal complications and the elimination of multiple operative procedures and/or radiologic interventional procedures is critical. The number of patients requiring dialysis continues to increase. Many of these patients are aged, obese, diabetic and often without adequate arteries or veins available for the construction of an AVF. Therefore, the standard IGAVF with its multiple complications is the only method available for providing vascular access to over 50% of patients requiring hemodialysis. In addition, if “home dialysis” or more frequent (daily) hemodialysis is ever to be realized, a simple, failsafe device for accessing the patient's circulatory system is essential. The elimination of the need to insert needles and the attendant complications may be important for such future systems.

### SUMMARY

Embodiments of the present invention provide devices and methods for maintaining permanent access to the patient's circulatory system, e.g., for the performance of hemodialysis (but any number of treatments requiring vascular access are envisioned). These embodiments provide a non-traumatic method for accessing a patient's circulatory system, providing high blood flows, and creating a simple, rapid and failsafe method for connecting to and disconnecting from the dialysis machine.

One embodiment consists of a single transcutaneous port implant which may be of various heights and diameters and which contains an open central cannel which may be of various diameters. The channel is closed by a “plug-in seal” when not in use. Attached to the port may be a tubular graft of PTFE material, which may be of various lengths and diameters. One end of the graft is anastomosed to an appropriate artery and the other end is anastomosed to an appropriate vein. Blood flows continuously through the graft at high volume and pressure. The graft is placed in a deep location within the patients' tissues and the port is also implanted in the patients' tissues and brought out perpendicularly through the skin. A cap may be employed to provide a sterile cover for the external exposed surface of the port implant and contained “plug-in seal”. When the implant port is in use the plug-in seal is removed and a double lumen tube is inserted into the open channel of the port by means of a sterile connector device. This tube completely fills the channel lumen and extends within the lumen of the attached PTFE graft partially occluding its lumen. The use of a specially configured double lumen tube to access the blood flow within the graft allows blood flow to and from the dialysis machine and minimizes recirculation of treated blood despite the use of a single entry site to the patients circulatory system. The connector device is employed to provide a sterile, secure method for removing the “plug-in seal” and inserting the double lumen tube to initiate dialysis; then at the completion of dialysis the tube is removed and a new sterile seal inserted. The connector device is small, sterile, simple to use and disposable.

The implant may have an external surface that promotes well-vascularized tissue ingrowth, by employing a mesh matrix and/or a porous configuration. This tissue ingrowth acts as a barrier to infection. In one embodiment, the surface material may be combined with an application of collagen and/or a silver polymer in order to create an additional barrier to infection. In addition the tissue ingrowth provides stability to the implant. The implant port and connector device provide a convenient, mechanical, sterile, and rapidly-deployable way to conduct dialysis.

The materials that may be used for the construction of the devices may be generally biocompatible. The surfaces exposed to blood flow will generally be non-thrombogenic.

Another embodiment includes two (2) implant ports substantially the same as the previously described implant port in configuration, material and surface. Each port however is attached to a circular or oval skirt of PTFE material which may be of various dimensions. The implant ports are individually anastomosed to an appropriately sized artery or vein with the graft skirt serving as a small patch sewn into the vessel wall and allowing access to the vessel lumen. When not in use the port channel is sealed and blood flows through the “patched” vessel. When in use a single lumen tube is inserted into the port channel and may or may not extend into the vessel lumen. Blood is removed from the arterial port and infused through the venous port. This arrangement allows placement of the ports at widely separated sites and eliminates recirculation and “steal” syndromes. Each port of the two port system requires a separate connector device of similar design to that used with the single port system.

In one aspect, the invention is directed toward a connector device for accessing an implant coupled to a graft forming part of a patient's vasculature. The device includes a housing including: an extraction assembly to remove a plug-in seal from an implant; a blood tube to access the vasculature; and an installation assembly including a new plug-in seal to insert the new plug-in seal into the implant; and at least one locking tab to lock the housing onto the implant.

Implementations of the invention may include one or more of the following. The graft may be a PTFE graft. The connector device may further include a cam dial, where rotation of the cam dial causes a distal movement followed by a proximal movement of at least one of the extraction assembly, blood tube, or installation assembly. The rotation of the cam dial may further cause a distal movement followed by a proximal movement of each of the extraction assembly, blood tube, and installation assembly. The rotation of the cam dial may cause driving posts attached to respective one of the extraction assembly, blood tube, and installation assembly to move distally and proximally along a helical barrel cam track. The installation assembly further comprising a locking pin and the locking pin may be configured to be inserted within a plug-in seal to secure the plug-in seal against movement within the implant. The connector device may further include a flush line to flush saline in a central passageway of the implant. The extraction assembly, blood tube, and installation assembly may be arranged within a turret, the turret rotating along with the cam dial.

In another aspect, the invention is directed toward an implant for accessing the vasculature of a patient. The implant includes a central cylinder, a locking flange coupled to the central cylinder at a proximal end thereof; an attachment mechanism coupled to a distal portion of the central cylinder, the attachment mechanism configured to attach the implant to a graft; and an ingrowth disk surrounding at least a portion of the central cylinder.

Implementations of the invention may include one or more of the following. The implant may be made of a material selected from the group consisting of: stainless steel, titanium, or combinations thereof. The locking flange may further include a protruding lip and a locking channel. The implant may further include a suture disk to secure the implant inside a patient, and the suture disk may be co-extensive with the ingrowth disk.

In yet another aspect, the invention is directed towards an implant for accessing the vasculature of a patient. The implant includes a central passageway, a locking flange coupled to the central passageway at a proximal end thereof; at least one horizontal passageway extending substantially perpendicularly to the central passageway, the horizontal passageway attached to the central passageway substantially at a distal end thereof; and an ingrowth disk surrounding at least a portion of the central passageway.

Implementations of the invention may include one or more of the following. The implant may be made of a material selected from the group consisting of: stainless steel, titanium, or combinations thereof. The locking flange may further include a protruding lip and a locking channel. The implant may further include another horizontal passageway extending substantially perpendicularly to the central passageway and in an opposite direction from the at least one horizontal passageway. The implant may further include a suture disk to secure the implant inside a patient, and the suture disk may be co-extensive with the ingrowth disk.

In yet a further aspect, the invention is directed toward a method of accessing the vasculature of a patient, including: attaching a connector device to an implant; locking the connector device onto the implant; extracting a plug-in seal from the implant; inserting a blood tube into the implant; removing the blood tube from the implant; installing a new plug-in seal into the implant; and removing the connector device.

Implementations of the invention may include one or more of the following. The method may further include priming the connector device. The method may further include flushing the implant with saline. The extracting a plug-in seal, inserting a blood tube, removing the blood tube, and installing a new plug-in seal, may be accomplished by rotating a cam dial.

The advantages of the invention may include but are not limited to one or more of the following: 1)needles are not required to be inserted either into an AVF or a IGAVF to gain access to the patient's circulatory system, thus potentially damaging the skin and subcutaneous tissues, as well as reducing pain and mental stress; 2) the implant port may be used immediately after implantation; 3) the implant port design, surface materials and connector design minimize the risk of infection; 4) a single implant port provides high blood flow rates to and from the dialysis machine, minimizes recirculation and minimizes red blood cell trauma; 5) the connector device may be rapidly attached to and removed from the implant port; 6) the plug-in seal may be removed and the double lumen tube inserted to initiate dialysis and the process reversed at the completion of dialysis placing a new sterile plug-in seal in place substantially automatically by means of the connector device, thus reducing human error and breaks in sterile procedure; 7) bleeding during the initiation, performance and completion of the dialysis procedure is reduced; 8) a double implant embodiment prevents steal syndrome and recirculation, and allows placement of implants at widely separated sites; 9) both single and double port implant embodiments can be placed deep within tissues adjacent to muscle for improved tissue ingrowth into the attached PTFE material and into well-vascularized tissue ingrowth into the port's mesh matrix or porous surface, and in addition sutures may be placed between the port's suture ingrowth disk or sewing ring or flange in order to combine to produce a stable system and to minimize infection; 10) home dialysis may be performed safely, easily, and sterilely by untrained personnel; 11) angiography, thrombectomy, angioplasty, and stenting, may be performed through the implant port, by removing the plug-in seal and inserting a valved connector to the implant port central channel; 12) only a small portion of the implant port need extend above the skin surface to allow placement of the connector device; 13) many of the embodiments of the system allow their use in patients with repeated failures of vascular access and in multiple locations not available to patients limited to the current methods of vascular access; 14) the system allows the use of anticoagulants without concern for bleeding from needle insertion sites; 15) the system can be placed deep within the patient's tissues using different implant heights, and requires minimal lengths and diameters of PTFE material for its use; 16) the system may be made in a low-cost fashion; and 17) the system reduces the unsightly appearance of a device in the patient's skin as only a small portion of the implant is visible, and may be easily covered with a dressing and/or the patient's clothing.

### BRIEF DESCRIPTION OF THE DRAWINGS

FIG. 1 is a schematic view of a graft-attached implant and connector device according to an embodiment of the present invention.

FIG. 2 is a schematic view of a graft-attached implant showing its interface with a patient's skin according to an embodiment of the present invention.

FIG. 3 is a perspective schematic view of an implant and connector device according to an embodiment of the present invention.

FIG. 4 is a perspective schematic view of an implant and connector device according to another embodiment of the present invention.

FIG. 5 is a perspective schematic view of an implant and graft according to an embodiment of the present invention.

FIG. 6 is an end-on view of the implant and graft of FIG. 5.

FIG. 7 is a side cross-sectional view of the implant and graft of FIG. 5.

FIG. 8 is a side cross-sectional view of an implant showing a plug-in seal and a locking pin in a first configuration.

FIG. 9 is a side cross-sectional view of an implant showing a plug-in seal and a locking pin in a second configuration.

FIG. 10 is a side cross-sectional view of an implant showing a plug-in seal and a locking pin with an extraction device.

FIG. 11 illustrates separated outflow and inflow lumens.

FIGS. 12(A)-(D) illustrate unitary or joined outflow and inflow lumens, also termed a dual lumen or bi-lumen tube, as part of a blood tube.

FIG. 13 illustrates a connector device and implant assembly according to an embodiment of the invention, joined to a graft, in a position to extract an indwelling plug-in seal.

FIGS. 14(A) and 14(B) illustrate side and top cross-sectional views of the connector device and implant of FIG. 13.

FIG. 15 illustrates the connector device and implant of FIG. 13, in a position having extracted the indwelling plug-in seal.

FIG. 16 illustrates the connector device and implant of FIG. 13, in a position to insert a blood tube.

FIG. 17 illustrates the connector device and implant of FIG. 13, in a position with a blood tube inserted through the implant to the graft.

FIG. 18 illustrates details of the driving post and mechanism by which the blood tube is inserted.

FIG. 19 illustrates the connector device and implant of FIG. 13, in a position to install a new indwelling plug-in seal.

FIG. 20 illustrates the connector device and implant of FIG. 13, in a position having installed the new indwelling plug-in seal.

FIG. 21 illustrates the connector device and implant of FIG. 13, showing a perspective external view.

FIG. 22 is a flowchart of a method of flushing the implant using the connector device prior to installation of a new plug-in seal.

FIGS. 23(A) and (B) illustrate views of a locking tab mechanism in a locked and unlocked configuration, respectively.

FIG. 24 illustrates an AV implant, according to an embodiment of the invention.

FIG. 25 illustrates an AV implant, according to another embodiment of the invention.

FIG. 26 illustrates a side-by-side embodiment of the connector device.

FIGS. 27(A)-(C) illustrate a gating device for use at a distal tip of a blood tube, according to an embodiment of the invention.

FIGS. 28(A)-(B) illustrate a spherical fluid gating device for use at a distal tip of a blood tube, in a partially-expanded configuration, according to an embodiment of the invention.

FIGS. 29(A)-(B) illustrate a spherical fluid gating device for use at a distal tip of a blood tube, in an expanded configuration, according to an embodiment of the invention.

FIG. 30 illustrates a flowchart of a method of use according to an embodiment of the invention.

### DETAILED DESCRIPTION

Embodiments of the invention are described below, generally involving an implant that accesses the vasculature and which may be in turn accessed by a connector device. First the implant is described, and then the connector device.

### Implant

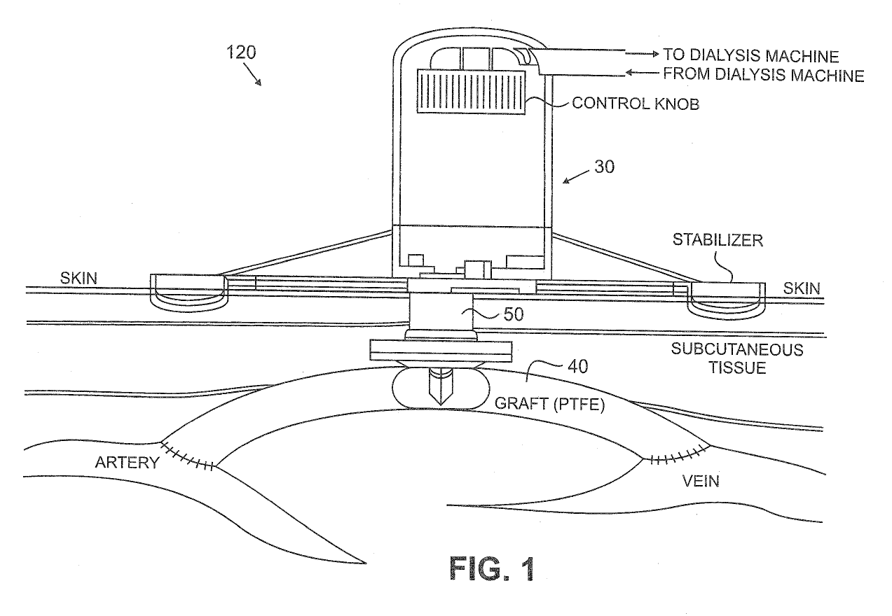

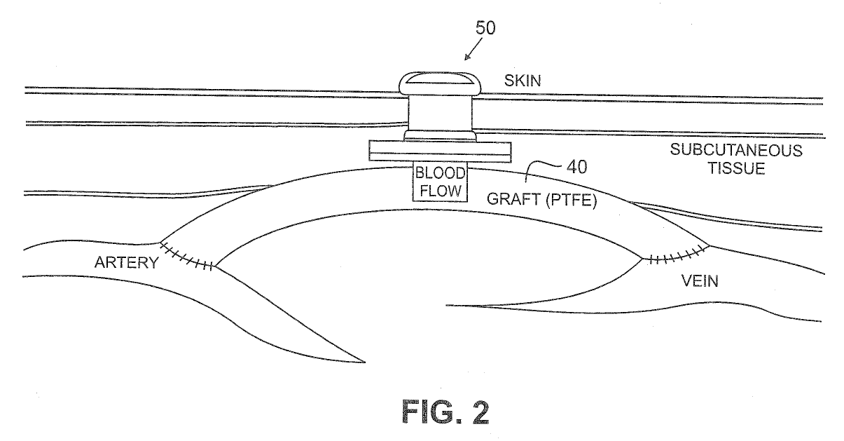

Embodiments are initially described of an implant system or assembly, also termed herein just an “implant”, which may be employed for a number of procedures involving vascular access. Referring to FIG. 1, the implant 50 may generally connect to a PTFE graft 40. Where the implant connects to a PTFE graft that extends between an artery and a vein, just one implant may be necessary; the one implant couples to a connector device 30 that houses and uses a dual-lumen blood tube. Where an artery and vein are used that are not connected by a graft 40, then two implants may be employed, one for the artery and one for the vein; in this system, two connector devices 30 are then employed, and each may house and use a single-lumen blood tube. In another embodiment, an artery and a vein can be pulled together for anastomosis, and a single implant employed that couples to each, this implant termed an “AV port”. This embodiment reduces the need for a graft between the artery and vein, and is described in greater detail below. The implant itself, without connection to a connector device, is illustrated in FIG. 2. These different implants, and associated connector devices, are discussed below.

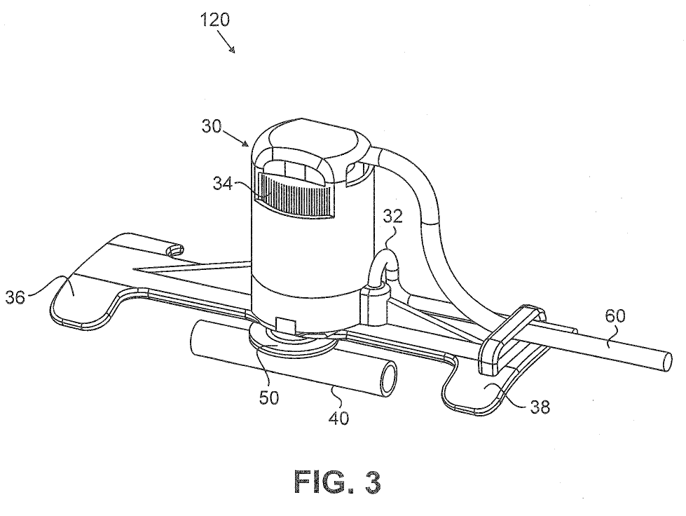

Referring to FIG. 3, a system 120 is described for the case where an implant 50 connects to a PTFE graft 40 that extends between an artery and a vein. The implant 50 also couples to a connector device 30 to allow a vascular procedure to be performed. One such vascular procedure that may be performed is a dialysis procedure. FIG. 3 also illustrates a dual lumen blood tube 60, a dial 34 for changing phases of the procedure, and stabilizers 36 and 38 for attaching the connector device 30 to a patient, e.g., a patient's arm. A saline flush line 32 is also shown.

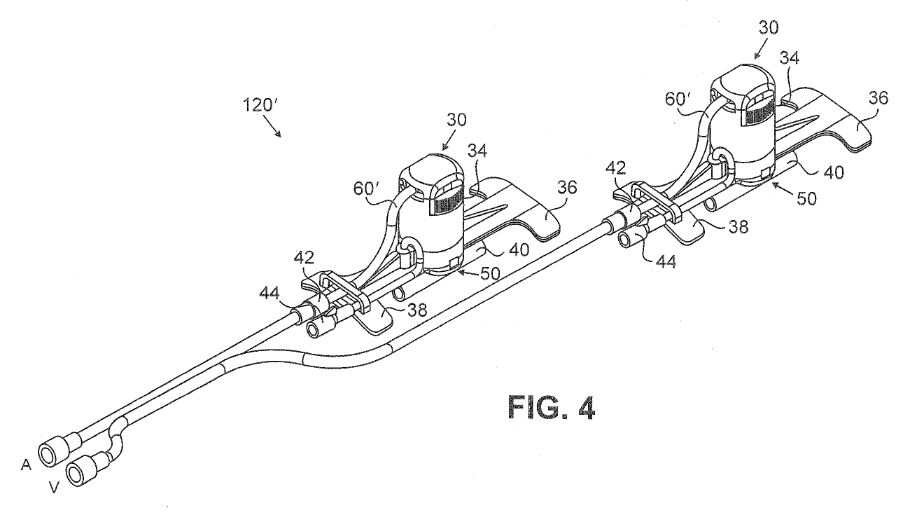

Referring to FIG. 4, a system 120′ is described for the case where an implant 50 connects to two PTFE grafts 40. One graft 40 forms a portion of an artery and another forms a portion of a vein. Each implant 50 also couples to a connector device 30 to allow a vascular procedure to be performed. FIG. 4 also illustrates a single lumen blood tube 60′. One implant is for arterial blood and the other for venous blood. A dial 34 may be employed for changing phases of the procedure, and stabilizers 36 and 38 for attaching the connector devices 30 to a patient, e.g., a patient's arm. Connectors 42 and 44 are illustrated, the former for connecting the venous and arterial sides to a procedure device, e.g., a dialysis machine, and the latter for connecting the saline flush line to a source of saline.

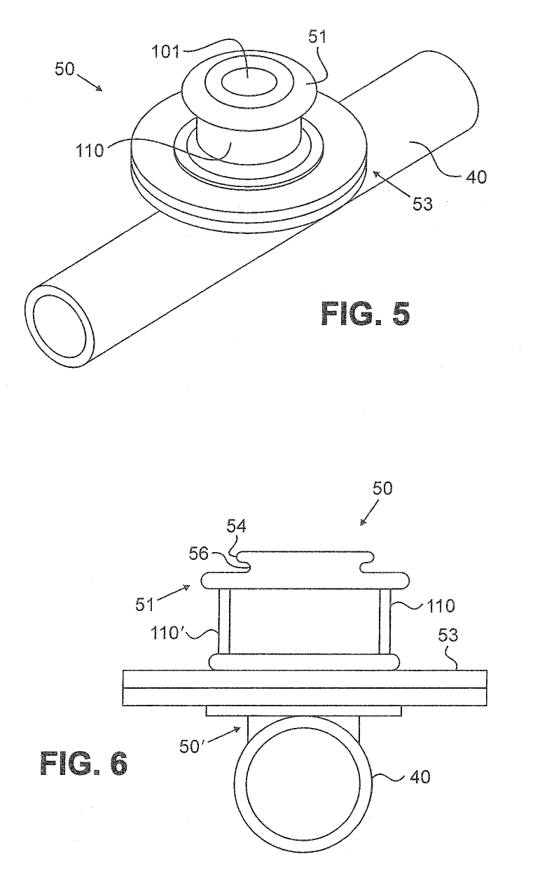

FIGS. 5-9 illustrate an exemplary implant 50 that is attached to a graft 40. The implant 50 includes a central cylinder 110 that defines a passageway through which the procedure is conducted. The passageway also defines where sealing occurs to prevent infection. Attached to a proximal end of the central cylinder 110 is a locking flange 51. Attached to a distal end of the central cylinder 110 is a suture ingrowth disk 53. A portion of the suture ingrowth disk 53 provides for ingrowth of biological material to enhance long-term stability, and another portion of the suture ingrowth disk 53 provides an optional location for attaching sutures for short-term stability. That is, sutures may be attached after implant installation to hold the implant in a fixed location, to the adjacent fascia, until ingrowth of biological material has occurred. Where the suture portion is made of a puncturable material, the same may be directly sutured into. Where the suture portion is made of a non-puncturable material, e.g., is made of metal, the same may define holes where sutures may penetrate. In some cases, the material of the suture ingrowth disk 53 may itself be biocompatible, and its shape and constitution may allow suturing. One appropriate biomaterial is one of the STARED materials available from Healionics, Inc. of Redmond, Wash. Alternatively, the material of the suture ingrowth disk may be coated with a biocompatible material. In another alternative, the suturing section is distinct, though may be connected, to the section that promotes ingrowth.

Tissue ingrowth into the external surface of the implant and into the PTFE graft material used for anastomoses to the blood vessels will, over a period of 14 to 28 days, significantly increase the stability of the device.

The suture ingrowth disk may be located at the junction of the proximal two-thirds and distal one-third of the implant length; the suture ingrowth disk extends from the implant's outer surface a distance of between about 2 and 15 mm, e.g., 8 mm, and has a thickness of approximately 1 to 10 mm, e.g., 5 mm.

The external surface of the implant and suture ingrowth disk may include a mesh matrix as described above, and which may be fabricated from the same material as the implant, and which may be, e.g., metallic or another suitable material. This surface of, e.g., 2-5 mm thickness, may include a porous structure with specific size interstices and material thickness and may have a texture that encourages vascularized tissue ingrowth.

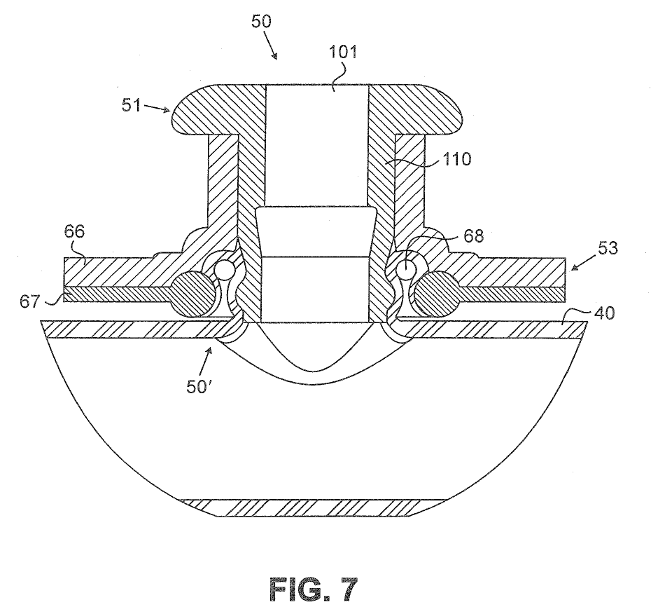

The upper edge of the external surface of the mesh matrix may be coated with a collagen layer that may be parallel to the skin surface and which may be positioned immediately below the level of the epidermis, encouraging epidermal growth over the surface and limiting or preventing the development of a sinus tract adjacent to the implant at its exit site through the skin. The area where ingrowth occurs may be not only on the disk but also on a central passageway 101, by way of the porous or mesh material extending not only over the disk but also in a cylinder around the central passageway (see element 110′ of FIG. 6). In this way, skin growth may occur in a direction directly parallel to the skin, straight into this porous or mesh or other such material, as well as into the disk itself. The porous or mesh material may be located above the disk 53, below the disk 53, or both.

FIG. 5 also illustrates the central passageway 101 through which procedures may be enabled by installation of a suitable catheter or other device. A sterile cap (not shown) may be employed to cover the system, although in most cases a plug-in seal is installed in a secure-enough fashion that the cap may provide only or primarily a cosmetic feature. The cap may be mounted onto the implant by way of an adhesive, by being threadingly screwed onto the implant, or via other techniques.

Referring to FIG. 6, the implant 50 is shown in cross-section, at an end-on view, i.e., at an angle looking in a direction parallel to a blood vessel. The graft 40 is shown attached to the implant 50. In this figure, the locking flange 51 is shown in greater detail, in particular showing a protruding lip 54 and a locking channel 56. The locking channel 56 may have a depth of, e.g., 30-90 mils, although other depths may also be employed according to the application. The protruding lip 54 may have a depth of, e.g. 10-50 mils, although other depths may also be employed according to the application. As will be described, one or more locking tabs engage the implant in the locking channel and cause a connector device to be secured to the implant because the locking tabs are locked within the locking channel 56.

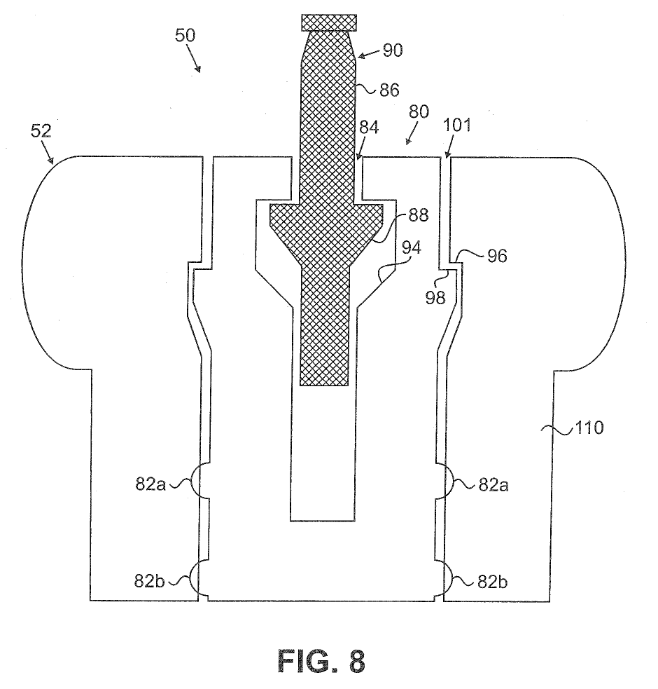

Referring to FIG. 7, a cross-sectional view of the implant 50 is illustrated. This view omits for clarity certain details of the locking flange 51, but shows the central passageway 101 which is defined by the central cylinder 110, as well as details thereof which assist in maintaining and securing a plug-in seal.

The suture ingrowth disk 53 is illustrated, and one potential arrangement of material constituent layers is shown for the suture ingrowth disk 53. In FIG. 7, a top layer 66 provides the suture attachment functionality, and the remaining layers act as a graft attachment mechanism 50′, to assist in the attachment of the implant to the PTFE graft. For example, a PTFE compression ring 67 may be employed to hold fast a section of the PTFE graft 40, in particular a section that is wrapped around a PTFE support ring 68. In this way, the implant may be secured to a portion of a PTFE graft.

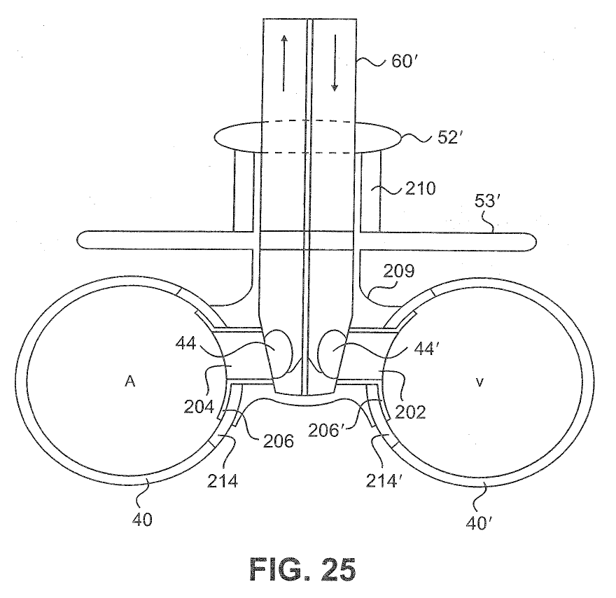

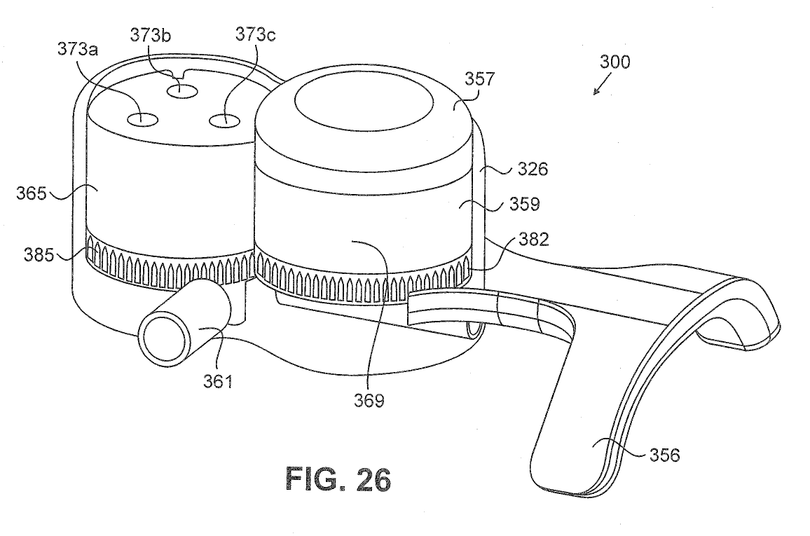

Other ways to secure the implant to the graft may also be employed. For example, a skirt may of a metal or a polymer, e.g., titanium, stainless steel, silicone, PTFE, polypropylene, or acetal, may be attached to a distal end of the central cylinder 110, the same for attachment to a graft. Other potential ways of attaching an implant to a graft are discussed below in connection with FIGS. 24 and 25, these also being employable to attach implant 50 to a graft.

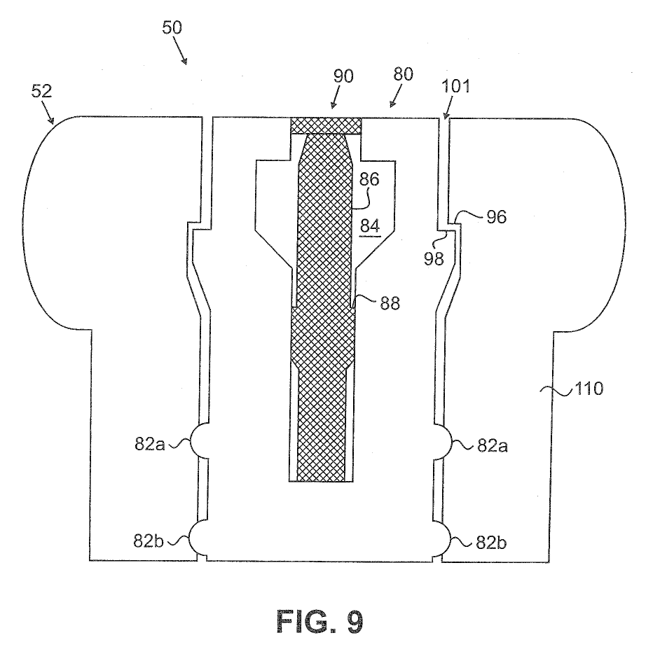

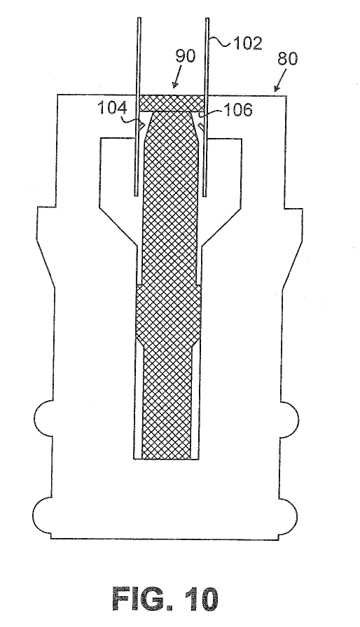

FIG. 8 illustrates details of an implant 50 in which a plug-in seal 80 is being installed. FIG. 9 shows the same, with the plug-in seal 80 fully installed. As is seen, the implant 50 has a locking flange 52 and an internal central passageway 101. The internal diameter of the central passageway 101 may be, e.g., 0.1 inches to 0.3 inches, although other diameters may be employed as needed according to the patient's size and vascular requirements.

In FIG. 8, the plug-in seal 80 has been installed but is not yet locked. The initial installation of the plug-in seal 80 has the plug-in seal 80 held in place by its locking ring 98 held by a ridge 96 defined on the implant 50. The plug-in seal 80 may also have ribs 82a and 82b that compress during installation and thereby further secure the plug-in seal 80 in a friction fit. The components are flexible, as described below, and so the plug-in seal 80 is secured but may still be removed. To lock the plug-in seal 80 in place, a locking pin 90 is employed. The locking pin 90 is inserted into a void 84 in the plug-in seal 80. In more detail, the locking pin 90 is mounted into the void 84 prior to use. During use, the same is fully inserted into the void 84 to securely lock the plug-in seal in the implant.

The locking pin 90 includes a generally cylindrical section 86 with a frustum 88 that flares or tapers out in a proximal direction from the cylindrical section 86. When the locking pin 90 is forced downward into the plug-in seal 80, the frustum 88 is forced into and against a corresponding frusta' section 94. When the frustum 88 is secured in this section, as shown in FIG. 9, the locking ring 98 can no longer flex away from the ridge 96. Accordingly, the plug-in seal 80 is held in the central passageway 101 in a very tight fashion, and can only be removed by a force significantly greater than that encountered in a blood vessel. The device and method by which the plug-in seal 80 is installed and removed and the locking pin 90 is installed and removed within the plug-in seal 80 is described below.

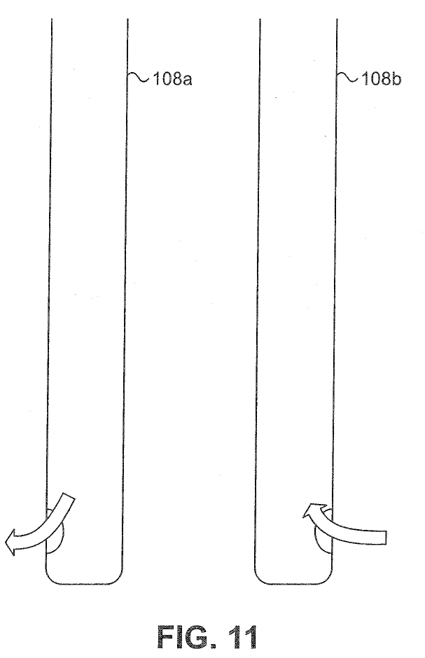

To remove the plug-in seal 80, an extraction device 102 shown in FIG. 10 may be employed. The extraction device 102 is controlled by the connector device, as will be described, and the same is extended in a distal direction and then retracted in a proximal direction to remove an indwelling plug-in seal 80. The extraction device 102 is generally cylindrical so as to surround the locking pin 90, although a partial cylinder may also be used. The extraction device 102 includes at least one feature with which to engage and remove the locking pin 90. For example, the extraction device 102 may include two tabs 104 that extend radially inwardly and proximally. These tabs act in a way similar to barbs, and when the extraction device is inserted distally far enough, the tabs 104 can engage an overhang 106 on the locking pin. By then moving the extraction device 102 proximally, first the locking pin 90 is removed, followed by the plug-in seal 80.

Before describing the connector device, general comments regarding the implant are now provided.

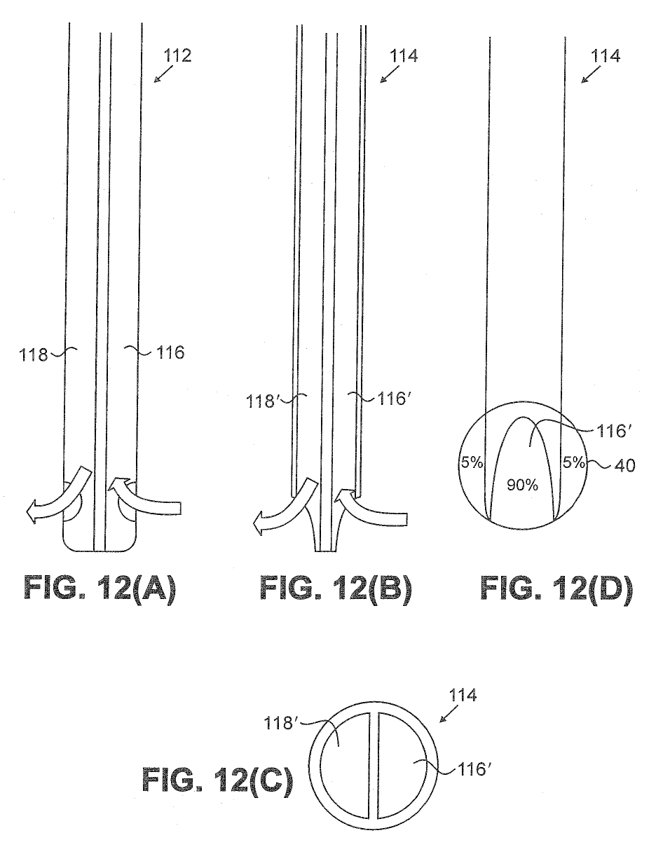

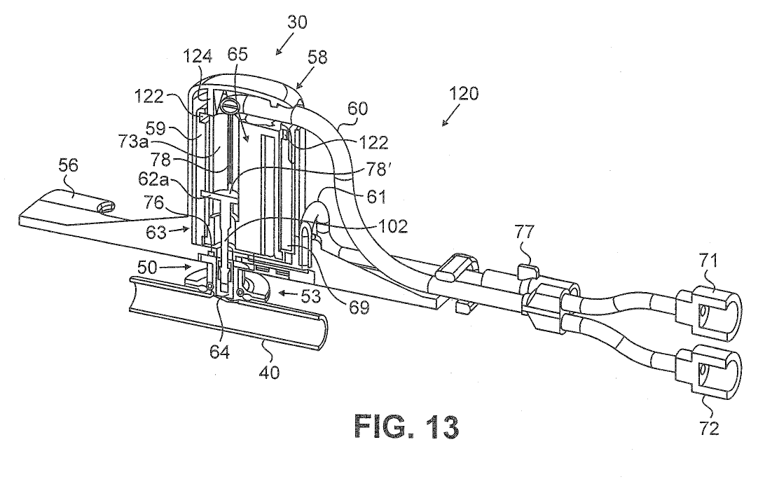

When the patient's circulation is to be accessed for connection to the dialysis machine, a single or dual-lumen blood tube may be moved downward, i.e., in a distal direction, a sufficient distance in order to allow a distal end of the blood tube to be inserted in the graft and to engage an opposite wall thereof. The blood tube is moved in the distal direction by the action of the connector device. Referring to FIG. 11, two single lumen blood tubes 108a and 108b may be employed where two implants are accessed, and a dual lumen blood tube (FIG. 12) may be employed in cases where just one implant is accessed.

Referring to FIG. 12, two varieties of dual lumen blood tubes are illustrated. In FIG. 12(A), skives may be made in a dual-lumen catheter blood tube 112 to define an outflow lumen 116 and an inflow lumen 118. In a dialysis vascular procedure, the outflow lumen 116 would direct the blood to a dialysis machine and the inflow lumen 118 would return the blood to the vessel from the same machine. In FIG. 12(B), skives and a tapered cut may be made in a dual-lumen catheter blood tube 114 to define an outflow lumen 116′ and an inflow lumen 118′. In some cases, depending on configuration, the blood tube of FIGS. 12 (B) and (C) may allow greater blood flow around the blood tube in order to provide, e.g., a washing or rinsing effect. As shown in the end-on view of FIG. 12(D), in which the graft 40 is also illustrated, the portion of blood transported out of the body by the blood tube may be, e.g., about 90% of the total blood flow in the vessel. The other 10% is directed around the blood tube to achieve a cleaning effect.

This configuration occludes, e.g., over 90% of the GAVF lumen and directs a large volume of blood flow into the outflow channel and substantially prevents the recirculation of blood returned from the dialysis machine through the inflow channel. The partial occlusion of the lumen allows a small amount of continuous blood flow around the blood tube and through the GAVF, providing a washing effect during dialysis.

The implant may be attached to tubular PTFE grafts of various diameters and lengths. The method of attachment of the graft to the implant may use the same technique whether for a single implant system or whether for a dual implant system. An elliptical opening of an appropriate shape and size to match the internal circumference of the implant may be defined and a sleeve employed that extends outward a distance, e.g., 5 mm from the opening, both of which may be constructed during manufacture of the tubular PTFE graft. The method of attachment described below provides a tight seal of the PTFE graft to the implant.

The tubular PTFE graft may be anastomosed by standard vascular surgical techniques to a suitable artery and vein, creating a functional GAVF. The combination of various implant lengths (heights) and graft diameters and lengths allows implantation of the system in various locations in the patient's body. In particular, the system may be placed at deeper locations dependent only on the length, i.e., the height, of the implant. The length used will depend on the patient's size, thickness of the subcutaneous tissues, and depth of the blood vessels to which the implant is attached. The external diameter of the implant may be approximately 1.5 cm. Its proximal end may be configured for placement of a cap that provides a sterile cover for the implant and its contained plug-in seal when not in use. The implant may extend above the surrounding skin approximately 1.0-4.0 mm.

The implant may be fabricated from a material that can withstand the repeated stresses placed upon it when the cap or connector device is placed and removed.

The implant at its lower or distal end may incorporate a lip, e.g., a 1 mm lip, to provide a way to attach the skirt of, e.g., PTFE graft material. The PTFE graft material may be elliptical in shape with the long axis oriented to the long axis of the vessel to which it will be anastomosed and the short axis oriented to the transverse diameter of the vessel to which it will be anastomosed. The skirt may be available in several dimensions appropriate to the size of the vessels to which it will be anasotomosed. The skirt may have a central opening substantially commensurate with the internal diameter of the implant, and a sleeve extending a distance, e.g., 5 to 10 mm, from the central opening. The sleeve may be stretched to fit over the lip at the distal end of the implant. The sleeve may be fixed to the implant with a tight circumferential wrap of multiple strands of monofilament PTFE thread of small diameter. The same may then secure the PTFE graft skirt to the implant and eliminate space between the PTFE graft skirt and the distal edge of the implant.

The skirt may be anastomosed to the blood vessel using a standard vascular surgical technique after excising a minimal elliptical portion of the wall of the vessel. This in effect widens the vessel at the interface between the implant PTFE skirt and the blood vessel. This configuration, in conjunction with the “washing” effect of repeated outflows and inflows of large volumes of blood through the implant, prevents the growth of tissue and/or thrombus across the interface.

When the implant is not in use, the plug-in seal made of blood-compatible materials may be placed within the lumen of the implant as a seal as has been described. The plug-in seal fits tightly within the implant and may have a smooth blood compatible surface. The fit of the plug-in seal within the implant and its smooth blood compatible surface assists in the prevention of thrombus formation at the interface.

A sterile cap of appropriate material may be seated securely over the upper surface of the implant and plug-in seal to preserve sterility and to prevent the plug-in seal from being inadvertently snagged by garments or the like. An antimicrobial circular gasket may be placed on the bottom surface of the cap that extends beyond the outer surface of the implant. To further enhance the stability of the implant, an optional ingrowth bowl 215 (illustrated in FIG. 24 in connection with an AV implant) may be employed, the same allowing surface epidermis tissue to radially terminate into the mesh structure, growing down into the ingrowth mesh and creating a tight vascularized infection barrier. A combination of porosity and weave network enables the creation of varying density configurations.

The disclosed implant systems provide small implants that have very little foreign body material exposed. The same are configured to be stable, both vertically as well as rotationally, allowing the surface interfaces to be safe and trauma-free. Ingrowth around the structures and through the ingrowth mesh at least in part is responsible for the stability of the systems.

### Connector Device

The connector device is a disposable catheter unit that is designed to initiate and terminate the desired functions for blood access. The same has a semi-automated design to allow the various functions to occur. These different modes of operation complete the entire blood access procedure, e.g., for dialysis. The connector device may employ a control dial that positions its components in specific ways into the implant for the procedure. One advantage may be that seating pressures and alignments may be automatic with this design, allowing constant, safe, infection-free and low manipulation of the implant and the connector device.

The connector device may accomplish the following five main objectives. First, it can be locked circumferentially and securely onto an upper surface of the implant. Next, it extracts and removes the plug-in seal positioned within the central passageway of the implant, into the housing of the connector device. Next, it inserts a blood conduit or tube from the connector device into the implant's central passageway a sufficient distance to create a partially obstructing gate within an attached PTFE graft. This conduit connects with the connector device's inflow and outflow tubing and provides separate channels for blood flow to and from the PTFE graft and a dialysis machine. Next, the connector device retracts the blood conduit from the central passageway of the implant at the completion of the procedure. Finally, the connector device installs a new sterile plug-in seal into the central passageway of the implant as the central passageway and top of the implant are flushed with saline from, e.g., a saline flush line in the connector device.

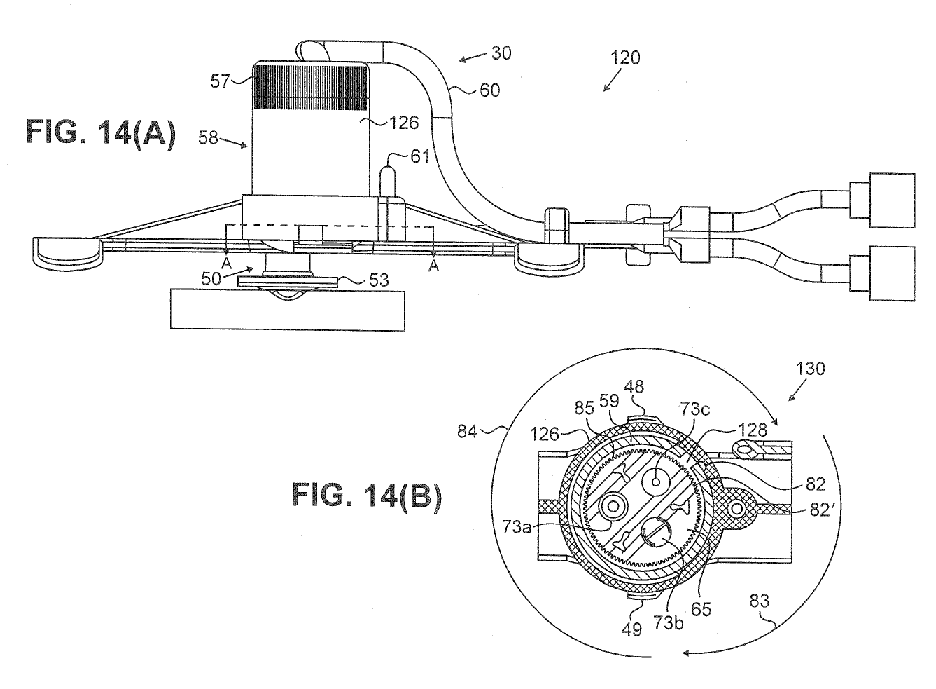

In more detail, and referring to FIG. 13, a system 120 includes a connector device 30 having a number of components. The connector device 30 is coupled to an implant 50, and it is understood that the implant 50 may accommodate a single or dual lumen blood tube 60. In FIG. 13, an interface seal 76 is illustrated that forms an airtight seal between the connector device 30 and the implant 50. Certain elements of the implant 50 are shown with the same reference numerals as noted above, including a suture ingrowth disk 53 and the implant's attachment to a graft 40.

The connector device 30 includes an arterial blood line 71 and a venous blood line 72, which combine to form an AV blood tube 60. The blood tube 60 may be open as shown, or enclosed in a protective cover (not shown) to prevent inadvertent operator interference. A connector 77 is provided for a flush line 61, the flush line 61 for delivering saline to the central passageway 101 of the implant. A stabilizer 56 may be provided on one or more sides in order to inhibit movement of the connector device 30. A dial assembly 58 forms the core of the device 30, and the same includes a rotating turret 65 which presents different subsystems to the implant 50 in a prescribed order.

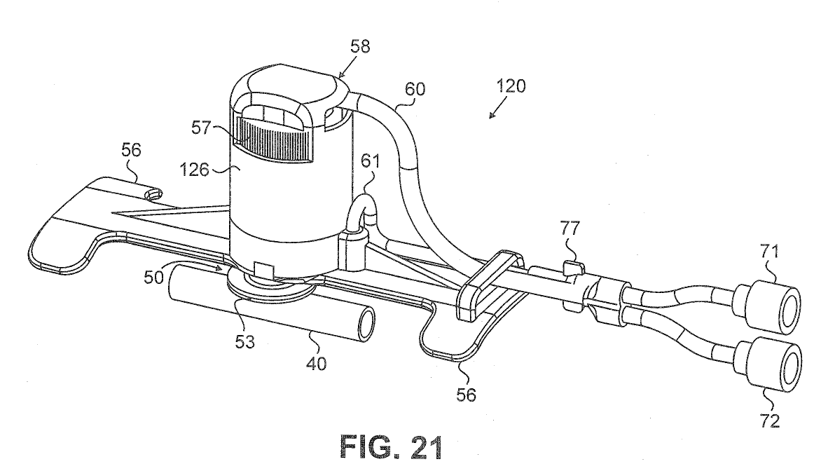

The turret 65 includes three internal cylinders 73a, 73b, and 73c (see FIGS. 13, 14(B), 15-17, and 19) each spaced generally at a 120-degree angle from each other. Each cylinder has at least one vertical groove 78 to guide the pistons up and down; and each piston has at least one guide post 78′ (see FIG. 20) which is inserted into, and which rides in, the corresponding vertical groove 78. The three cylinders each have a different purpose for the procedure, e.g., a dialysis procedure. For example, the first cylinder extracts the plug-in seal from within the implant's central passageway in a manner described below. This vertical movement is repeated within all three cylinders in order to lower and raise their contained assemblies or tubes as the cylinder housing rotates during the use of the connector device at the start, during, and at the completion of the procedure. Conveniently, all internal components stay within the connector device, and the turret and cam dial may be made to rotate in only one direction.

Each cylinder encloses a separate assembly, and each assembly has a piston with a driving post 62a, 62b, and 62c, which allows a cam to cause vertical movement. The driving posts for each piston ride in a top track 122 until such time as an internal cam cylinder, driven by the cam dial, contacts an engagement pin 124, at which point the driving post is pushed down (by riding down the engagement pin 124) into a helical barrel cam track 69. The helical barrel cam track 69 forces the driving post (and thus the piston) first down in a distal direction and then up in a proximal direction. Between the down and up movements is a travel distance or stall mode, e.g., for 120 degrees, where the piston is going neither up nor down.

In the position shown in FIG. 13, a plug-in seal 64 is about to be extracted from the implant 50 using a plug-in seal extraction assembly 63, which includes a piton 63′ and an extraction device 102. The extraction piston 63′ and the extraction device may be inserted around the plug-in seal 64 by the action of the connector device 30, in particular by operating or rotating a turret cylinder 73a into position (this position may be a default or initial position, i.e., the position when shipped) and rotating the cam dial, thereby causing a driving post 62a to traverse down a helical barrel cam track 69 (of course, it is the helical barrel cam track 69 that rotates). As the plug-in seal extraction piston 63′ is constrained by the guide track 78 and guide post 78′, it has only one degree of freedom, i.e., up or down, and the same is forced downward into the plug-in seal 64. As described above, the extraction device 102 may be employed to engage and, upon movement in a proximal direction, remove the plug-in seal 64 from the implant 50.

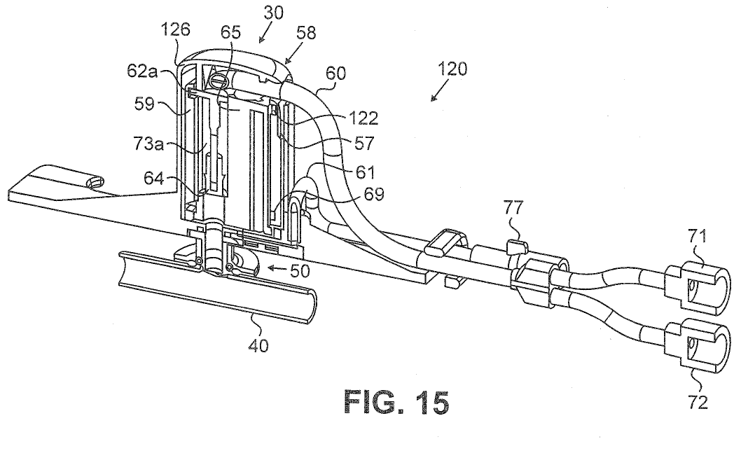

In more detail, and referring in addition to FIGS. 14(A) and 14(B), the system 120 and connector device 30 are illustrated (FIG. 14(B) is a cross-section along lines A-A of FIG. 14(A)). A cam dial 57, shown with serrations for ease of turning, is a constituent part of the dial assembly 58. The cam dial 57 is integral with an internal cam cylinder 59, on the inside cylindrical wall of which is the helical barrel cam track 69. By rotating the cam dial 57, an operator may cause one or more pistons to move up or down within one of the three cylinders 73a, 73b, or 73c, disposed within the turret 65.

The rotation drives these pistons, and the rotation may be configured to be in the same direction by use of an appropriate clutching mechanism discussed below, e.g., a clutch cam device 130 shown in FIG. 14(B). The internal cam cylinder 59 is discontinuous with discontinuity 128, and the cylinder 59 further includes a head section 82 with a spline section, e.g., radially inward-facing teeth 82′. The head section 82 is cammed, e.g., bulbous or otherwise extended in a radially outward direction. In this way, the head section 82 may engage a corresponding ramp on the interior of the cylinder 59, the ramp extending, e.g., 120 degrees, although many variations of the amount of ramp may be provided. When the head section 82 engages the ramp, the head section 82 is bowed inward such that the teeth 82′ engage a spline on the turret 65, e.g., teeth 85 on the exterior of the turret 65. When they are so engaged, cam dial rotation moves the vertical driver cam 59 and the turret 65 together for a 120-degree displacement 83 for the next cylinder, then releases for a 240-degree rotation 84, this 240-degree rotation being employed for vertical movement of the cylinder pistons. Like with the 120-degree rotation, the 240-degree rotation may vary significantly, e.g., a 5-degree overtravel (or more or less) may be provided. Each 120-degree rotation may be temporarily arrested by way of a detent.

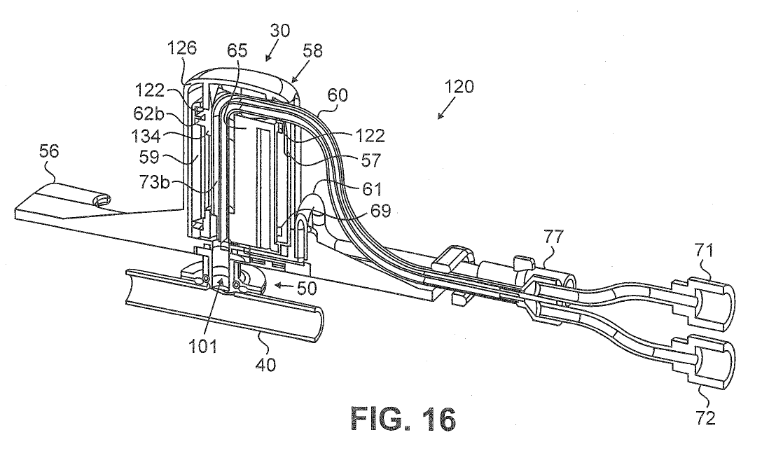

Returning to the sequential movement of the turret 65, FIG. 15 illustrates the connector device 30 and implant 50 following a turret rotation in which the rotation has caused the extraction of the plug-in seal 64. In a way opposite to that in which the extraction assembly 63 was engaged onto the plug-in seal 64, the rotation moves the extraction assembly 63 in the upward (proximal) direction because the same causes the driving post 62 to traverse up the helical barrel cam track 69.

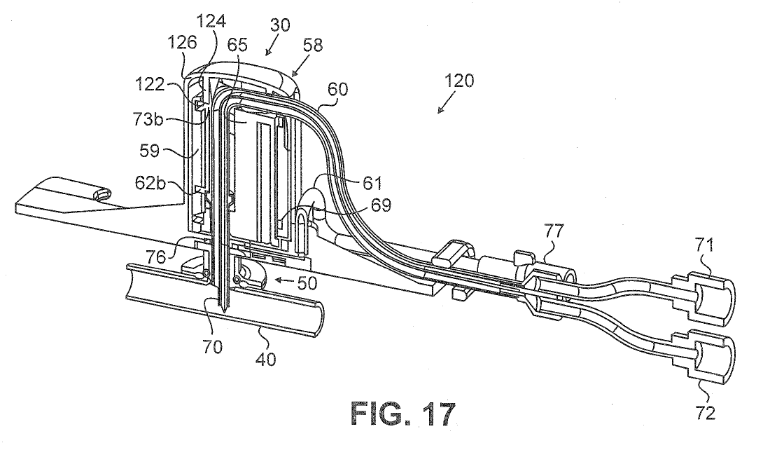

FIG. 16 illustrates the next sequential stage of rotation. In FIG. 16, the extraction assembly 63 and plug-in seal 64 have been rotated away from the implant 50 and in their place is the AV blood tube 60 in a raised position. It will be understood that, in a two-implant system, the blood tube 60 need only have or employ a single lumen, while in a single implant system, the blood tube 60 will have two (or more) lumens. In FIG. 16, the AV blood tube 60 is in a retracted position. Other details of the AV blood tube have been discussed above.

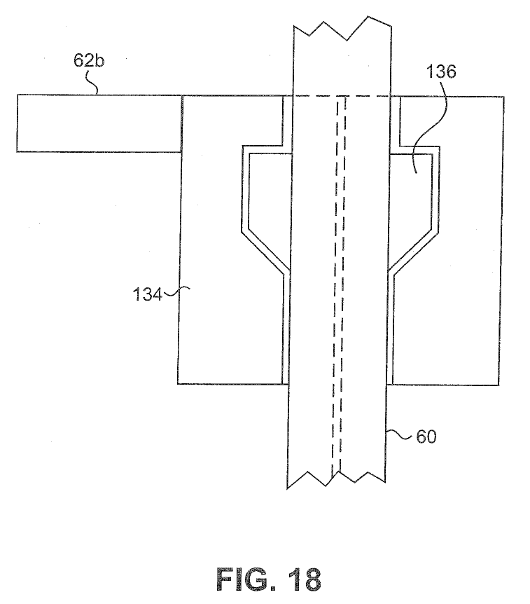

As with the plug-in seal extraction, the driving post 62b is forced down the helical barrel cam track 69 via rotation of the internal cam cylinder, forcing the blood tube 60 through the cylinder 73b and into the implant 50 as illustrated in FIG. 17. The rotation causes the insertion in the same way as the descending engagement of the plug-in seal extraction assembly. The blood tube 60 may be generally provided with enough slack to allow the blood tube to be inserted into the graft as well as enough slack to accommodate the rotation itself.

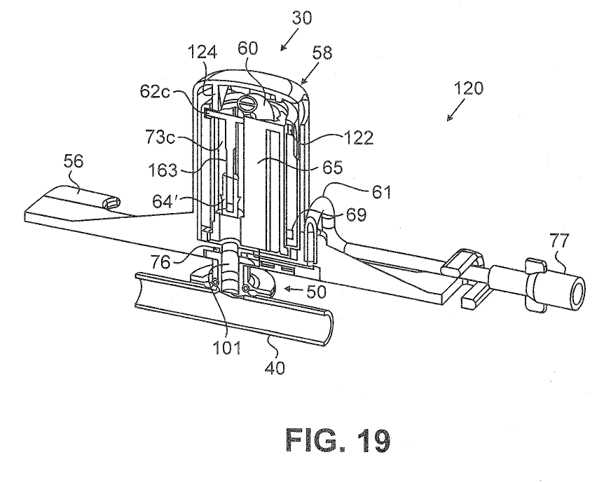

The configuration of the driving post 62b is substantially similar to that of driving posts 62a and 62c; however, what the same attaches to is somewhat different since the blood tube 60 requires a continuous lumen throughout the cylinder 73b, unlike the situation with the plug-in seal extraction and insertion. Referring to FIG. 18, a driving post 62b is illustrated as attached to a driving torus 134. The driving torus defines an inner void in which may be disposed a blood tube driving annulus 136. Using such a configuration, movement of the driving post 62b can result in movement of the blood tube 60.

After insertion, the procedure, e.g., dialysis, may be conducted. During the procedure, the dial assembly 58 may be in the stall mode.

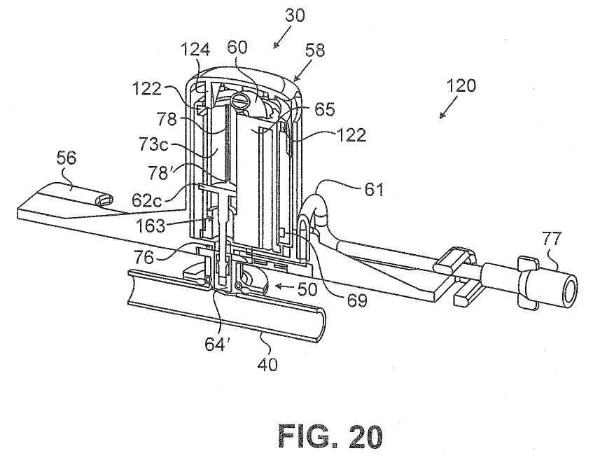

Following the procedure, the blood tube 60 is retracted and rotated away from the implant 50, again via a cam dial and turret rotation. In particular, FIG. 19 illustrates the connector device 30 following another rotation of the turret 65. In this orientation, the blood tube 60 has finished accessing the vasculature, and has been rotated away from the implant 50. In its place is another cylinder 73c, this cylinder 73c containing an installation assembly including a new unused and sterile plug-in seal 64′ mounted on a plug-in seal installation piston 63′. By rotating the turret 65 in the same direction as before, the seal installation piston 63′ may be forced in a downward direction via the post 62c having the helical barrel cam track 69 rotating about the same, and thus sealing the implant 50. The installed position is illustrated in FIG. 20. As discussed above in connection with FIGS. 8 and 9, installation of the plug-in seal 64′ is accomplished not only by inserting the same into the implant but also by inserting a locking pin 90 into the plug-in seal 64′.

Retraction of the plug-in seal installation piston 63′ is accomplished in the same way as removal of the plug-in seal extraction piston 63, by rotation of turret 65.

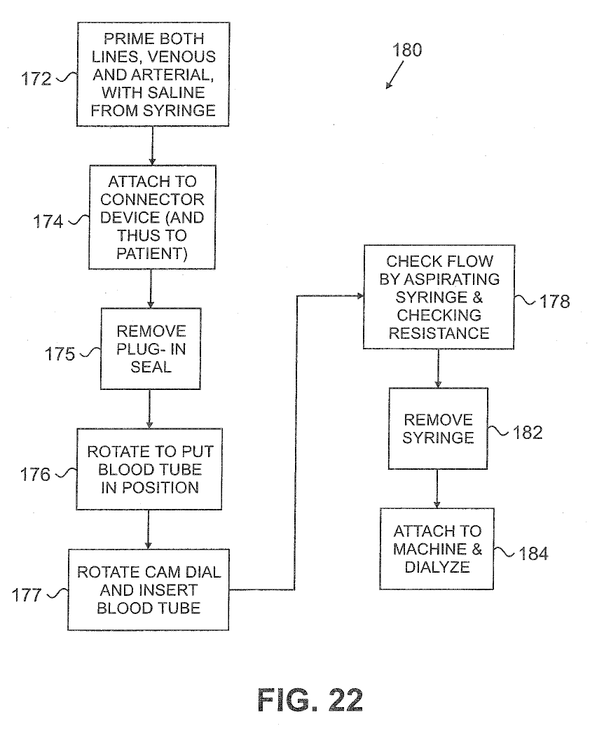

FIG. 21 illustrates a perspective view of the system 120 including the connector device 30 coupled to the implant 50.

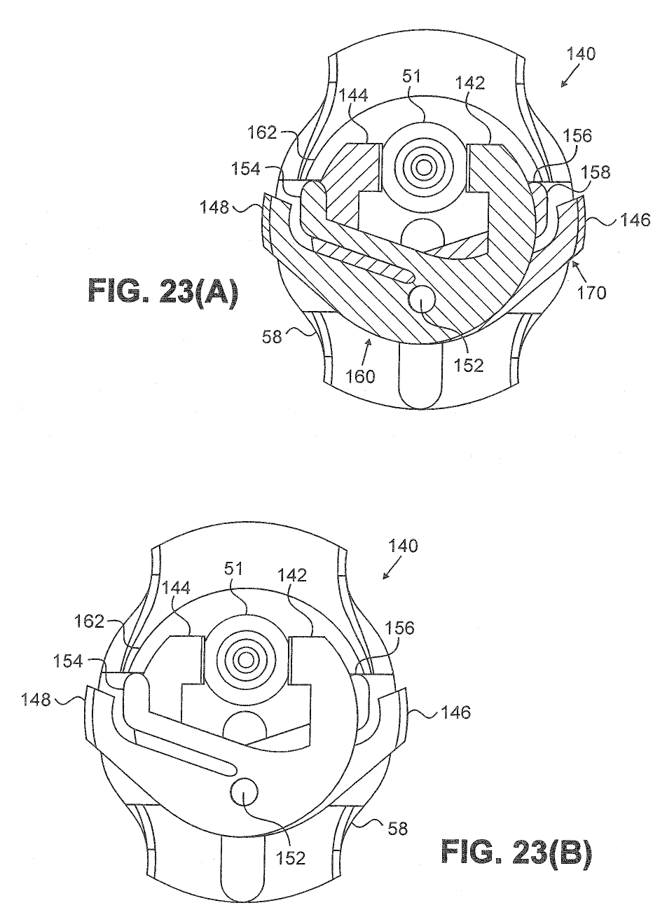

Termination of the dialysis may include a sterile saline flush as the plug-in seal 64′ is inserted into the implant central passageway 101. The flush line 61 infuses the saline in order to rinse all blood components from the implant central passageway 101. In more detail, and referring to the flowchart of FIG. 22, a first step in the method 180 is to prime both lines to be attached to the connector device 130, i.e., the arterial and venous sides, with saline from, e.g., a syringe (step 172). A next step is to attach the lines to the connector device 130 and thus to the patient (step 174). A next step is to rotate the turret in the manner described above so as to remove the plug-in seal (step 175) and further rotate the turret in the manner described above to put the blood tube above the implant central passageway 101 (step 176). Further rotation inserts the same into the implant (step 177). A next step, to ensure there is no clotting of the implant, is to check the blood flow by aspirating or pulling back on an inserted syringe (the syringe may be inserted into the manifold to which the arterial and venous lines attach (step 178). Assuming no clotting, the syringe is removed (step 182) and the arterial and venous lines may be attached to a dialysis machine for a dialysis procedure (step 184).

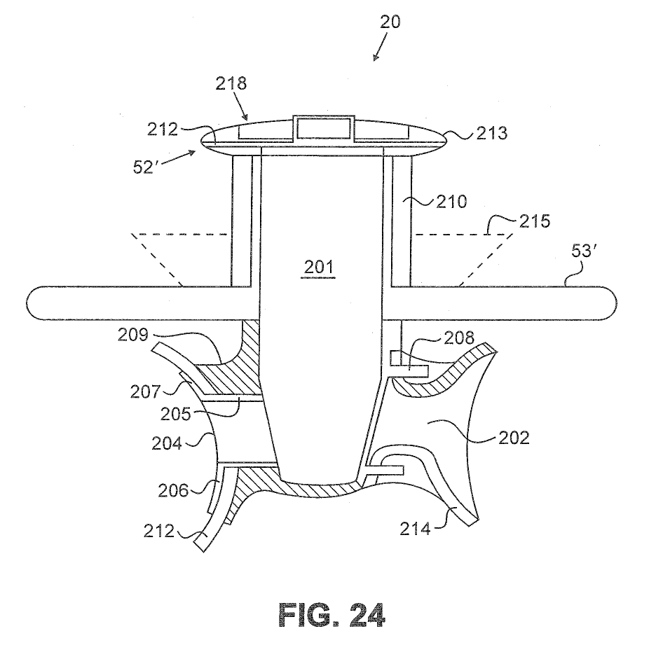

FIGS. 23(A) and 23(B) illustrate a locking mechanism which may be employed to lock the connector device 30 onto an implant 50. Of course, it will be understood that other locking mechanisms may also be employed. In particular, FIG. 23(A) illustrates the locking mechanism in a rest configuration, in which the same is locked onto an implant locking flange 51. The locking mechanism includes a first locking tab 160 and a second locking tab 170 which rotate around a pivot 152. The locking tabs may be, e.g., 10-40 mils thick, although other thicknesses may also be employed. The first locking tab 160 includes a finger tab 148, a flange engagement tab 142, and a tensioner 154. The tensioner 154 engages an abutment 162 to bias the locking tab 160 in a counterclockwise direction. The second locking tab 170 includes a finger tab 146, a flange engagement tab 144, and a tensioner 158. The tensioner 158 engages an abutment 156 to bias the locking tab 170 in a clockwise direction. By an operator squeezing the finger tabs 148, the biases are overcome and the flange engagement tabs 142 and 144 rotate away from the locking flange 51, as shown in FIG. 23(B), allowing the connector device to be removed from the implant. Of course, the same technique allows the connector device to be attached to the implant.

### Variations

### AV Implant

An AV implant system may be employed, in another embodiment, and used in place of the implant 50. The AV implant system requires that one implant be joined to an appropriately-sized artery and a second implant joined to an appropriately-sized vein in a manner described below. The implants generally do not attach directly to arteries and veins but rather attach to grafts, or artificial vessel portions, which have been installed surgically. Sometimes, an entire vessel portion is replaced with a graft. Other times, just a portion of the vessel is replaced, e.g., just an elliptical portion, so as to allow the implant to achieve a substantial purchase on the vessel. In some cases, a circular annulus of graft material, e.g., PTFE, may be wrapped around an element on the implant, allowing the blood to only contact PTFE or the interior of the implant, minimizing the chance of infection or other maladies.

Referring to FIG. 24, an AV implant system 20 is illustrated that is an implanted structure made of, e.g., titanium, but which may be made of any biocompatible material. The AV implant system 20 is a small three-way structure with two horizontal passageways and a vertical lumen 201 that travels upward (or in a proximal direction) toward the surface tissue where the implant protrudes with an external implant interface. The system 20 may include a locking flange 52′ to which a connector device 30 may be attached in the same manner as that of implant 50 above.

The vertical lumen 201 is defined by a central cylinder 210, and allows access by a catheter to two separate vascular grafts, one which forms part of an artery and one which forms part of a vein. The catheter in turn may have two channels, e.g., may be a split-channel catheter.

The AV implant system 20 includes two horizontal lumens 202 and 204, these lumens 202 and 204 defined by walls 206 and 208, respectively, which are attached to skirt segments 212 and 214, respectively. The skirt segments are coupled to or mounted to the arterial and venous grafts. The skirts may be made of, e.g., PTFE. In this way, the skirts can anastomose to the artery and vein, replacing a portion of the body's vessel wall. The skirts become covered with the body's natural endothelial cells, which in time become part of the vessel lumen.

FIG. 24 shows the AV implant system 20 with two different ways of attaching to grafts, while FIG. 25 shows the AV implant system 20 with two of the same ways of attaching to grafts.

In FIG. 24 (on the left hand side of the implant), a wall device 206 includes a cylindrical section 205 which mates with a hole in the central cylinder 210. This mating may be by way of a press-fit. At a distal end of the cylinder 205 is a frusto-conical section 207. The PTFE skirt 103 may be made elastic enough to allow a hole or sleeve section of the same to be moved over the frusto-conical section 207. The hole or sleeve section is then trapped between the frusto-conical section 207 and a wall 209 of the AV implant 20. By further pressing the cylinder 205 into the hole of the central cylinder 210, the PTFE sleeve becomes incapable of removal.

Also in FIG. 24 (on the right hand side of the implant), a PTFE skirt is illustrated as attached to the implant 20 via another technique. A sleeve 214 of the PTFE skirt is inserted and wrapped around an element 208 and held in place by a friction fit, by another ring, or by puncturing the sleeve with a pin formed integrally with the implant. Other ways will also be seen given this teaching. These configurations may create more of a natural lumen surface, exposing less titanium to the blood stream.

FIG. 25 shows insertion of a blood tube 60′. Arterial blood flows into a blood tube 60′ by way of an arterial port 44, and flows back into the patient's vasculature following dialysis by way of a venous port 44′, these ports forming the two aforementioned horizontal lumens. FIG. 25 also illustrates wall 206′, which functions in a way similar to wall 206, and which couples to a skirt section 214, which is in turn anastomosed to a graft 40′.

FIGS. 24 and 25 show other elements, these similar to those of implant 50, and indicated by primed components. For example, a suture ingrowth disk 53′ is illustrated, which as before may include a titanium mesh with a treated coating and/or a circular support such as for suturing. FIG. 24 also illustrates various other components, which may also be employed in implant 50. These include a seal seat 218 and locking tabs 212 and 213, which may be employed in other types of locking configurations. An ingrowth bowl 215 may be optionally included, the same allowing tissue to more gently fill in around the implant as ingrowth occurs.

A distal end of the central passageway 201 is a location at which the arterial and venous ports intersect, and may incorporate a slight taper, e.g., of between 2 and 10 degrees, e.g., 5 degrees. This taper assists in sealing the lumen, e.g., with a plug-in seal, as well as sealing the split-channel or dual lumen catheter 60′ when the same is installed for a vascular procedure such as for dialysis.

### Side-by-Side Design

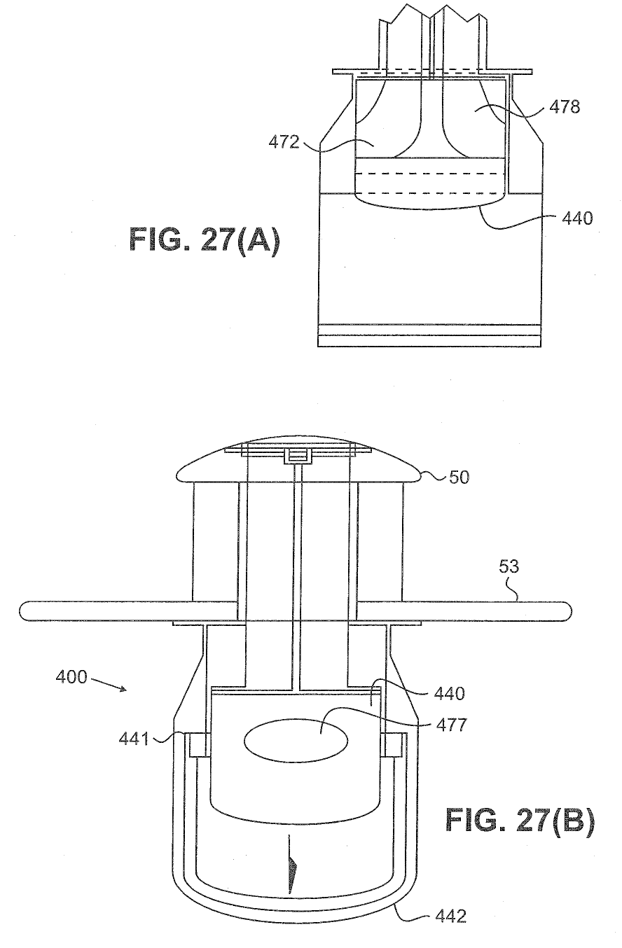

Referring to FIG. 26, a side-by-side connector device design is illustrated which may be useful when the vertical height of the turret is desired to be lessened. Referring to this figure, a connector device 300 includes an outside housing 326 and a stabilizer 356. A cam dial 357 is integral with a cylinder 359, which has an accompanying spline 382. Rotation of the spline 382 rotates a spline 385 on a turret 365. The turret 365 includes three cylinders 373a, 373b, and 373c. Rotating the cam dial 357 performs similar functions as in the design of connector device 30. In particular, splines 382 and 385 can be engaged or disengaged to rotate the turret, and rotation of the cam dial 357 also causes rotation of a helical barrel cam track 369, moving pistons within cylinders 373a, 373b, and 373c to move up and down. In this case, the driving posts for the cylinders are exterior of the cylinders, and extend to the cylinder 359 so as to engage a track 369 within the same.

### Gating Assemblies

In many cases, structuring the blood tube in accordance with FIG. 12 is enough to ensure sufficient blood flow is sent to dialysis and sufficient blood flow is available for flushing the implant during the procedure. In some cases, however, a small implant is installed in a large blood vessel, and in these and other cases it becomes necessary to gate the flow so that appropriate flow is achieved for both purposes.

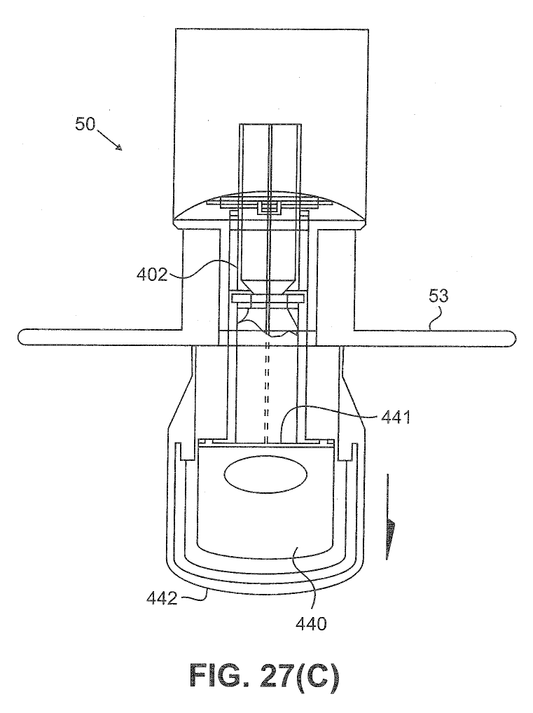

Referring to FIG. 27(A)-(C), a vascular gate 400 is illustrated which descends into the interior of the blood stream in the region of the graft. This gating is accomplished with a single piston 440 that can descend and occlude approximately 90% of the blood flow during dialysis. The gate has an arterial channel 478 with entry hole 477 on one side and a venous port 472 with an exit hole (not shown) on the other. A retracted configuration is shown in FIG. 27(A) and a partially deployed configuration is shown in FIG. 27(B).

This cylinder gate 400 intersects the graft at a distal end 442 which also has a cylindrical shape, and blocks off the majority of the blood flow. These two intersecting cylinders conform to the gate because the outside collar of the implant changes the shape of the graft in this location. This changes the orientation from cylindrical in a horizontal sense to cylindrical in a vertical sense. This sliding gate design is a high tolerance mechanism that reduces or eliminates leakage of blood through any adjacent channels, generally by means of an automated flushing system (see port 441 in FIG. 27(B)) which is designed to fill the space and prevent blood from traveling between the housing and the gate piston. This auto-flush acts as a fluid bearing and keeps the respective surfaces clean.

The gate configuration may be slightly out-of-round, and enables the gate to maintain proper positioning, and eliminates the need for additional guides to prevent the gates from rotating. The lower portion of the gate has a large diameter, and the upper section has a smaller diameter which occupies the main passageway of the implant structure. This acts as an actuator device which allows the connector device to manipulate the gate. The actuation of the gate implant device is accomplished by the connector device, which locks onto the gate implant and positions an internal port system to interface with the gate implant. The positioning may be accomplished in the same manner as the descending of any of the aforementioned assemblies. FIG. 27(C) illustrates the fully-extended configuration.

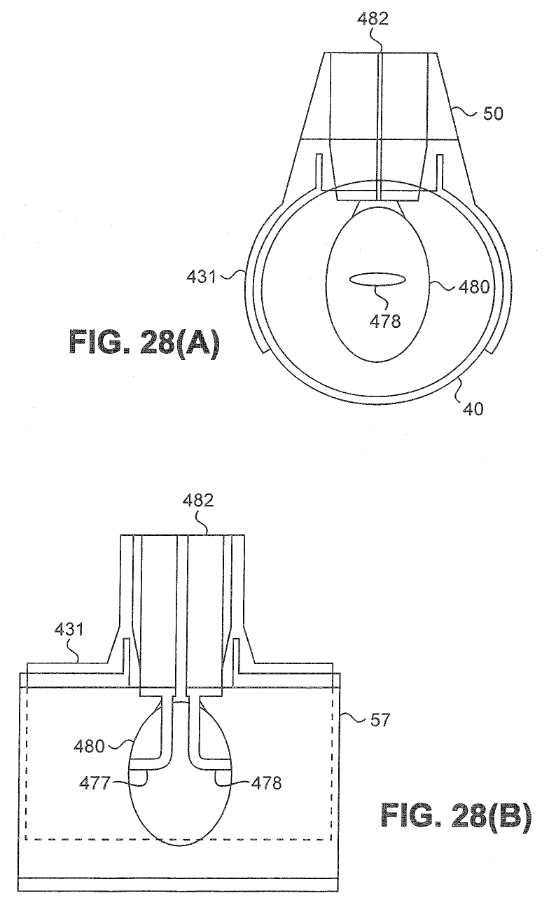

FIGS. 28(A)-(B) and 29(A)-(B) illustrate another gate assembly, this embodiment including a fluid bladder and in particular a spherical fluid gate. Fluid flexible bladder systems may be employed to inflate and gate the blood flow in the vessel to prevent arterial and venous mixing. The bladders may be formed of rubber or the like and may be filled with saline for expansion and inflation.

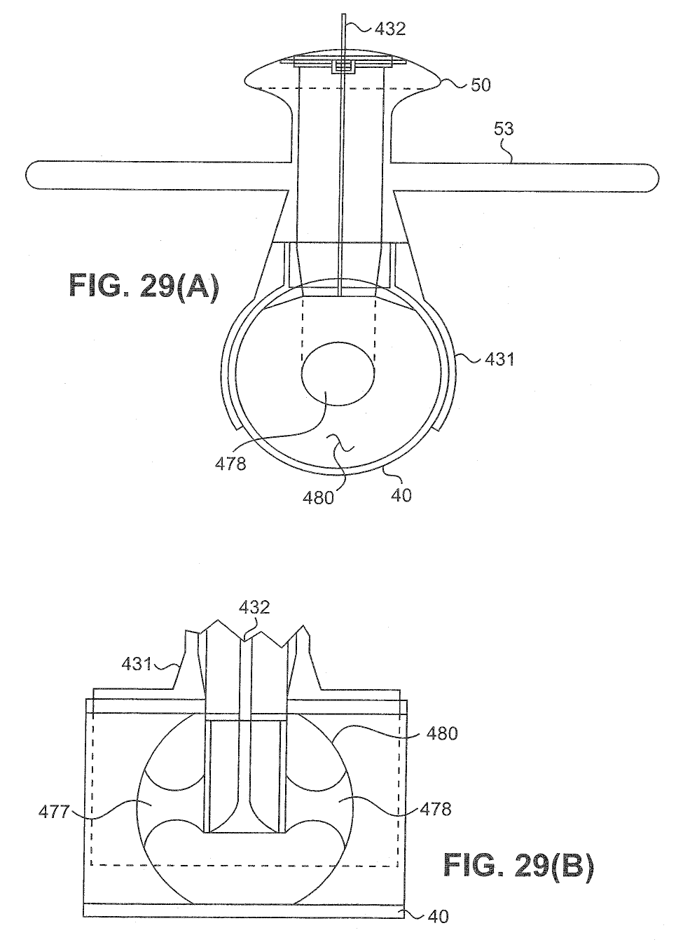

FIGS. 28(A)-(B) illustrate the gate during expansion and FIGS. 29(A)-(B) illustrate the gate after expansion. A spherical balloon 480 is inflated via a fluid inflation passageway 432, which may be valved to the saline flush line 61. When inflated, lumens 477 and 478 are defined for arterial and venous blood. The remainder of the blood vessel or graft, or a substantial portion thereof, is occluded. Other elements are also shown of implant 50, including a suture ingrowth disk 53. An optional support collar 431 is shown, which may provide additional support to the PTFE graft 40.

This configuration allows for a spherical ball to dominate the interior of the blood vessel. Saline is injected, e.g., from the connector device, into the implant via the fluid inflation passageway above. The preformed arterial and venous passageways expand open until the sphere has inflated. The attachment of the sphere to the distal portion of the catheter area allows the rigid section to port from the outer perimeter to the interior of the catheter. The ports molded from the catheter allow arterial blood flow into the connector device and back to the opposite side of the sphere.

In an alternative embodiment, a structure may be placed around the balloon so as to direct the balloon inflation in specific directions, e.g., to more effectively occlude the vessel. For example, the balloon may be between two parallel plates, each with a window formed therein. During inflation, the balloon may be configured to expand through the windows, thereby tending to occlude the vessel in a planar fashion. If passageways are formed in the balloon leading to a blood tube, the same may be effectively employed in a vascular procedure.

In yet another embodiment, a vascular ring bladder design may be employed which expands a fluid bladder from the exterior of a vascular ring which surrounds the vessel or graft. The external balloon applies pressure to the outside of the vessel, closing around the positioned catheter and narrowing the blood flow through the area. A portion of the vessel wall may then drop down through a portion of the support ring that is open. An injection of saline, to inflate the bladder, travels through the structural housing of the implant from the connector device.

### Shape Variations

Implants may differ in many ways; e.g., implants for use with dual-lumen blood tubes may have more of an elliptical shape in their central passageway or graft sleeves, as well as a larger proximal, i.e., upper, surface area to accommodate both the outflow and inflow blood channels present.

One potential purpose of the elliptical shape for the implant is to maintain the correct orientation of the implant and its outflow and inflow channels, as well as any gate and plug-in seal, when the implant is in use. One correct orientation of the plug-in seal may be such that its long diameter is perpendicular to the direction of blood flow within the GAVF. This allows occlusion of the GAVF, providing maximum flow rates and preventing re-circulation.

The distal or lower openings may be larger than the proximal openings and may also be elliptical. The elliptical shapes of the openings and the large channels result in maximum blood flow volumes with lessened turbulence.

A central portion of the plug-in seal may include a gate and may be continuous with an attached foot. The foot is generally thin, flexible, elliptical in shape, and of sufficient surface area to cover and seal the site of the implant attachment to the GAVF. The plug-in seal also may cover and seal the lower openings of the outflow and inflow channels when they are not in use.

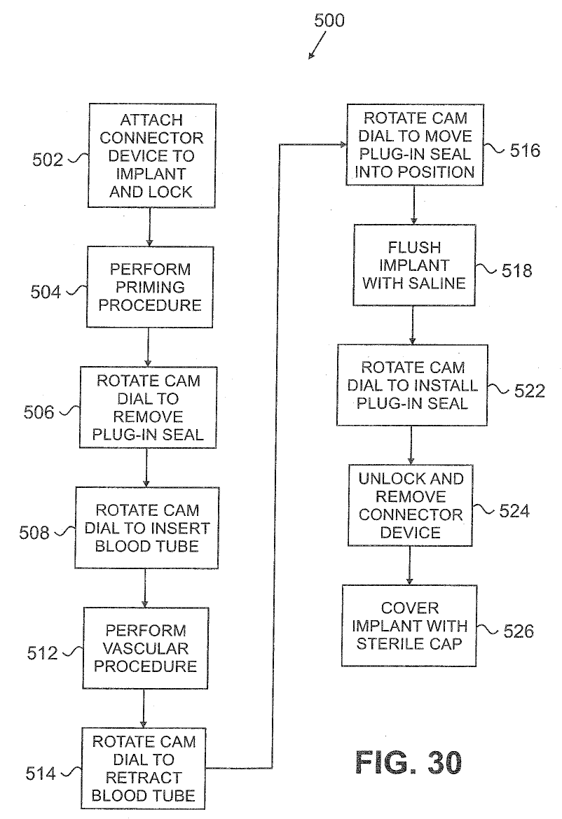

Referring to the flowchart of FIG. 30, a method 500 according to an embodiment of the invention is illustrated. A first step is to attach the connector device to the implant and to lock the two together by way of the locking tabs or via another variety of locking mechanism (step 502). A next step is to perform a priming procedure as described above (step 504). Steps 502 and 504 may be switched if desired. A next step is to rotate the cam dial so as to extract a plug-in seal (step 506). A next step is to rotate the cam dial so as to move a blood tube into position and to further rotate the cam dial so as to insert the blood tube (step 508). A vascular procedure, e.g., dialysis, may then be performed (step 512). The cam dial is then rotated so as to retract the blood tube (step 514). The cam dial is then rotated so as to move a new plug-in seal into position for installation (step 516). The implant is then flushed with saline (step 518) so as to minimize the risk of infection as well as to reduce the chance of air bubble inclusion upon insertion of a new plug-in seal. Following the flush, the cam dial may then be rotated so as to insert the plug-in seal (step 522). The connector device may be unlocked and removed (step 524). An optional final step is to cover the implant with a sterile cap (step 526).

The materials employed in the connector device and implant may be as follows, although other materials will also be understood to be employable. The material of the turret, internal cylinder having the helical barrel cam track, as well as plug-in seal extraction device and insertion device, may be delrin, nylon, or other polymer materials. The plug-in seal may be, e.g., silicone, and the locking ring may be made of delrin or the like. The support and suture ring may be, e.g., silicone as well as metals such as titanium. The housing of the connector device may be, e.g., polycarbonate. O-rings may be, e.g., silicone or the like. The saline flush line may be, e.g., PVC. The locking pin may be made of various metals, e.g., stainless steel or the like. The locking tabs may be made of spring steel, e.g., 17-7 spring steel, brass alloys, polymeric materials, or the like. In general, where two components are in contact or moving against one another, they should be of different materials. One of ordinary skill in the art will recognize that other materials may also be employed.

The above description has been with respect to certain specific embodiments. The invention, however, is not to be limited to those specifics. Accordingly, the invention is to be limited solely by the claims appended hereto, and equivalents thereof.

1. A connector device for accessing an implant coupled to a graft forming part of a patient's vasculature, comprising:
a. a housing including:
i. an extraction assembly to remove a plug-in seal from an implant;
ii. a blood tube to access the vasculature; and
iii. an installation assembly including a new plug-in seal to insert the new plug-in seal into the implant; and
b. at least one locking tab to lock the housing onto the implant.

2. The connector device of claim 1, wherein the graft is a PTFE graft.

3. The connector device of claim 1, further comprising a cam dial, wherein rotation of the cam dial causes a distal movement followed by a proximal movement of at least one of the extraction assembly, blood tube, or installation assembly.

4. The connector device of claim 3, wherein rotation of the cam dial causes a distal movement followed by a proximal movement of each of the extraction assembly, blood tube, and installation assembly.

5. The connector device of claim 4, wherein rotation of the cam dial causes driving posts attached to respective one of the extraction assembly, blood tube, and installation assembly to move distally and proximally along a helical barrel cam track.

6. The connector device of claim 1, wherein the installation assembly further comprising a locking pin, and wherein the locking pin is configured to be inserted within a plug-in seal to secure the plug-in seal against movement within the implant.

7. The connector device of claim 1, further comprising a flush line to flush saline in a central passageway of the implant.

8. The connector device of claim 1, wherein the extraction assembly, blood tube, and installation assembly are arranged within a turret, the turret rotating along with the cam dial.

9. An implant for accessing the vasculature of a patient, comprising:
a. a central cylinder;
b. a locking flange coupled to the central cylinder at a proximal end thereof;
c. an attachment mechanism coupled to a distal portion of the central cylinder, the attachment mechanism configured to attach the implant to a graft; and
d. an ingrowth disk surrounding at least a portion of the central cylinder.

10. The implant of claim 9, wherein the implant is made of a material selected from the group consisting of: stainless steel, titanium, or combinations thereof.

11. The implant of claim 9, wherein the locking flange further comprises a protruding lip and a locking channel.

12. The implant of claim 9, further comprising a suture disk to secure the implant inside a patient.

13. The implant of claim 12, wherein the suture disk is co-extensive with the ingrowth disk.

14. An implant for accessing the vasculature of a patient, comprising:
a. a central passageway;
b. a locking flange coupled to the central passageway at a proximal end thereof;
c. at least one horizontal passageway extending substantially perpendicularly to the central passageway, the horizontal passageway attached to the central passageway substantially at a distal end thereof; and
d. an ingrowth disk surrounding at least a portion of the central passageway.

15. The implant of claim 14, wherein the implant is made of a material selected from the group consisting of: stainless steel, titanium, or combinations thereof.

16. The implant of claim 14, wherein the locking flange further comprises a protruding lip and a locking channel.

17. The implant of claim 14, further comprising another horizontal passageway extending substantially perpendicularly to the central passageway and in an opposite direction from the at least one horizontal passageway.

18. The implant of claim 14, further comprising a suture disk to secure the implant inside a patient.

19. The implant of claim 18, wherein the suture disk is co-extensive with the ingrowth disk.

20. A method of accessing the vasculature of a patient, comprising:
a. attaching a connector device to an implant;
b. locking the connector device onto the implant;
c. extracting a plug-in seal from the implant;
d. inserting a blood tube into the implant;
e. removing the blood tube from the implant;
f. installing a new plug-in seal into the implant; and
g. removing the connector device.

21. The method of claim 20, further comprising priming the connector device.

22. The method of claim 20, further comprising flushing the implant with saline.

23. The method of claim 20, wherein the extracting a plug-in seal, inserting a blood tube, removing the blood tube, and installing a new plug-in seal, are accomplished by rotating a cam dial.

24. A set of implants for accessing the vasculature of a patient, comprising:
a first implant including:
a central passageway;
a locking flange coupled to the central passageway at a proximal end thereof;
at least one horizontal passageway extending substantially perpendicularly to the central passageway, the horizontal passageway attached to the central passageway substantially at a distal end thereof, the horizontal passageway of the first implant accessing an artery of a patient; and
an ingrowth disk surrounding at least a portion of the central passageway; and
a second implant including:
a central passageway;
a locking flange coupled to the central passageway at a proximal end thereof;
at least one horizontal passageway extending substantially perpendicularly to the central passageway, the horizontal passageway attached to the central passageway substantially at a distal end thereof, the horizontal passageway of the second implant accessing a vein of a patient; and
an ingrowth disk surrounding at least a portion of the central passageway;
such that blood may be removed from the patient using the first implant, treated by a dialyzer, and returned to the patient using the second implant.

25. A method of accessing the vasculature of a patient, comprising:
attaching a first connector device to a first implant;
locking the first connector device onto the first implant;
extracting a plug-in seal from the first implant;
inserting a blood tube into the first implant;
attaching a second connector device to a second implant;
locking the second connector device onto the second implant;
extracting a plug-in seal from the second implant;
inserting a blood tube into the second implant;
removing blood using the first blood tube, delivering the blood to a dialyzer, and delivering blood from the dialyzer to the patient using the second blood tube;
removing the blood tubes from the respective implants;
installing new plug-in seals into the respective implants; and
removing the connector devices.
